(19) United States
(12) Patent Application Publication
(10) Pub. No.: US 2024/0156711 A1
(43) Pub. Date: May 16, 2024

(54) PICKERING EMULSIONS STABILIZED BY CYCLODEXTRIN

(71) Applicant: Firmenich SA, Satigny (CH)

(72) Inventors: Wolfgang FIEBER, Satigny (CH); Pascal BEAUSSOUBRE, Satigny (CH); Addi FADEL, Plainsboro, NJ (US)

(73) Assignee: Firmenich SA, Satigny (CH)

(21) Appl. No.: 18/552,042

(22) PCT Filed: Mar. 25, 2022
(86) PCT No.: PCT/EP2022/057878

§ 371 Date: Sep. 22, 2023

Related U.S. Application Data
(60) Provisional application No. 63/167,196, filed on Mar. 29, 2021.

(30) Foreign Application Priority Data
May 10, 2021 (EP) 21173135.1

(51) Int. Cl. A61K 8/73; A61K 8/06; A61L 9/012; A61Q 13/00; A61Q 19/00

(52) U.S. Cl. CPC A61K 8/738; A61K 8/06; A61L 9/012; A61Q 13/00; A61Q 19/00; A61K 2800/52

(57) ABSTRACT

This invention relates to a liquid product in the form of an oil in water or a water in oil emulsion stabilized by a cyclodextrin, methods for obtaining the emulsions, consumer products, and uses thereof.

### CROSS REFERENCE TO RELATED APPLICATIONS

The present application claims priority to U.S. Provisional Application No. 63/167,196, filed on Mar. 29, 2021, and European Application No. 21173135.1, filed May 10, 2021. The entire contents of these applications are explicitly incorporated herein by this reference.

### FIELD OF THE INVENTION

This invention relates to a liquid product in the form of an emulsion stabilized by a cyclodextrin, methods for obtaining the emulsion, and uses thereof.

### BACKGROUND

There is an increasing demand for reduced or ethanol free products in applications such as fine fragrance. This demand has increased in the post-Covid19 period. In the past, such applications have been hampered by problems such as skin irritancy.

### SUMMARY OF THE INVENTION

The present invention meets the industry-recognized need for ethanol free formats by providing emulsions stabilized by a cyclodextrin.

The present invention provides an emulsion comprising: an aqueous phase comprising water, an oil phase comprising a perfume oil and an oil-miscible solvent, and a stabilizer selected from the group consisting of: an α cyclodextrin, a β cyclodextrin, a γ cyclodextrin, a hydroxy-propyl modified cyclodextrin, a methyl modified cyclodextrin, or combinations thereof.

An oil-miscible solvent according to the present invention may be, for example, tributyl-O-acetylcitrate, triethylcitrate, caprylic triglyceride, triacetin, coconut alkanes (and) coco-caprylate/caprate, propanediol dicaprylate, octanoic acid 1,3-propanediyl ester, isopropyl palmitate, isopropyl myristate, ethyl oleate, triheptanoin or mixtures thereof.

In certain aspects, the oil-miscible solvent is between 1% and 70% by weight based on the total weight of the emulsion, or between 10% and 60% by weight based on the total weight of the emulsion.

An emulsion of the present invention may further comprise an oil-miscible co-solvent. The oil-miscible co-solvent may be, for example, caprylic/capric glycerides, undecane and tridecane, C15-C19 alkanes, squalene, a silicone oil, a glycol ether such as tripropylene glycol methyl ether, dipropylene glycol n-propyl ether, DIPG monomethyl ether, a dimethyl adipate/dimethyl glutarate ester, benzyl benzoate or mixtures thereof.

The oil-miscible co-solvent may be between 1% and 50% by weight based on the total weight of the emulsion, or between 5% and 40% by weight based on the total weight of the emulsion.

A stabilizer of the present invention may be, for example, an α cyclodextrin, a β cyclodextrin, a γ cyclodextrin, a hydroxy-propyl modified cyclodextrin, a methyl modified cyclodextrin, or combinations thereof.

In aspects of the present invention, the α cyclodextrin, β cyclodextrin, γ cyclodextrin, hydroxy-propyl modified cyclodextrin, methyl modified cyclodextrin, or combination thereof is between 0.001% and 10%, between 0.001% to 1%, or between 0.001% to 0.5% by weight of the emulsion.

In some aspects of the present invention, the water phase does not include a C2-C4 alcohol.

An emulsion of the present invention, in some aspects, may further comprise an oil-miscible weighting agent in the oil phase. The oil-miscible weighting agent may be, for example, a gum, an estergum, a damargum, sucrose acetate isobutyrate, or a combination thereof.

In some aspects, the oil-miscible weighting agent may be between 5% and 50% or between 5% and 30% by weight based on the total weight of the emulsion.

In aspects of the present invention, the water phase comprises an oil-miscible viscosifier. The oil-miscible viscosifier may be, for example, a cellulose derivative such as ethylcellulose, polyethylmethacrylate polymer, bis-isobutyl PEG-24/PPG-7/dimethicone copolymer and mixtures thereof. In some aspects, the oil-miscible viscosifier is between 1% and 30% by weight of the oil phase or between 2% and 10% by weight of the oil phase.

In some aspects of the present invention, the water phase further comprises a water-miscible viscosifier. The water-miscible viscosifier may be, for example, xanthan gum, a natural gum (such as agar gum, alginic acid, gellan gum, tragacanth gum), guar gum, a cellulose derivative, polyacrylic acid, an acrylate copolymer, or a combination thereof.

In certain aspects, water-miscible viscosifier is between 0.05% and 5%, between 0.05% and 1% by weight of the water phase or between 0.05% and 0.3% by weight of the water phase.

An emulsion of the present invention may further include a water-miscible co-solvent. The water-miscible co-solvent may be, for example, a C₂-C₄ alcohol, ethanol, propylene glycol, hexylene glycol, dipropylene glycol, glycerol, isopropylidene glycerol, butylene glycol (1,3-butanediol), 1,2-pentanediol, 1,2-hexanediol, 1,3-propanediol, and isopropanol, ethyl lactate or a mixture thereof. In some aspects, the water-miscible co-solvent may be between 1 and 50% by weight of the water phase or between 2% and 20% by weight of the water phase. The water-miscible co-solvent may be of synthetic and/or natural origin.

An emulsion of the present invention may be a water-in-oil or an oil-in-water emulsion.

Emulsions of the present invention may comprise water phase droplets dispersed in the oil phase and be about 10% to about 80 wt % of the emulsion.

Emulsions of the present invention aspects wherein the oil phase is droplets dispersed in the water phase and comprises from about 10% to about 80 wt % of the emulsion.

In aspects of the present invention, the perfume oil in the oil phase is from 1% to 50 wt % of the emulsion or 5% to 20 wt % of the emulsion.

According to the present invention, the dispersed phase droplets may have a diameter from about 1 micron to about 8 millimeters. In other aspects, the dispersed phase droplets have a diameter from 0.1 to 5 mm. In yet other aspects, the dispersed phase droplets have a diameter 0.5 to 5 mm.

In some aspects of the present invention, the oil phase further comprises an apolar cosmetically acceptable solvent, an emollient, or a combination thereof.

In aspects of the present invention, the emulsion includes a surface modifying agent such as, for example, a surfactant, an amino acid, lecithin, an acid, an alcohol, a salt, and a combination thereof. Surface modifying agents of the present invention may include, for example, arginine, lysine, oleic acid, stearic acid, SDS, CaCl₂, or a combination thereof.

In an aspect of the present invention, the perfume oil comprises between 0.01 and 50% of ingredients having a logP of less than 2.5. In a further aspect, the perfume oil comprises between 0.01 and 50% of ingredients having a logP of less than 2.0. In another aspect, the perfume oil comprises between 0.01 and 50% of ingredients having a logP of less than 1.5.

The present invention encompasses a consumer product including the emulsion of the present invention. The consumer product may be, for example, a fine fragrance product, a body care product, or an air care product.

The present invention further includes use of the present invention for stabilizing water and oil emulsions.

### DETAILED DESCRIPTION

The present invention meets the industry-recognized need for reduced or ethanol free formats by providing oil in water emulsions and water in oil emulsions. Emulsions of the present invention are stabilized by solid particles. Emulsions of the present invention may optionally be devoid of surfactants or emulsifiers. Stable two-phase fine fragrance systems are provided by carefully choosing cyclodextrins according to the present invention.

In an aspect of the present invention, oil droplets are stabilized in a water phase by cyclodextrins (solid-stabilized “Pickering” emulsion). In another aspect of the present invention, water droplets are stabilized in an oil phase.

The solid-stabilized emulsions of the present invention:
may optionally be free of surfactant;
may be free of a C2-C4 alcohol;
may contain up to 80% of dispersed phase by weight of the emulsion; and
may including up to 50% by weight of perfume.

An O/W emulsion according to the present invention, stabilized by cyclodextrins, allows oil droplets of up to 5 mm to be dispersed in water. Droplets of such size give a solution an appealing visual effect to consumers of fine fragrances, body care products and air care products.

Surprisingly, giant oil droplets in water may be obtained according to the present invention.

A “perfume oil” or “fragrance oil” according to the present invention encompasses any synthetic or natural oil suitable for fragrance uses.

“LogP” is the logarithm of the octanol-water partition coefficient, which is known as a measure of lipophilicity.

Oil-miscible solvent: An emulsion composition of the present invention comprises an oil-miscible solvent, which may be, for example, tributyl-O-acetylcitrate, triethylcitrate, caprylic triglyceride, triacetin, coconut alkanes (and) coco-caprylate/caprate, propanediol dicaprylate octanoic acid 1,3-propanediyl ester, isopropyl palmitate, isopropyl myristate, ethyl oleate, triheptanoin, caprylic/capric glycerides, and mixtures thereof. In one embodiment, the oil-miscible solvent is triethylcitrate, tributyl-O-acetyl citrate and mixtures thereof. When present, the oil-miscible solvent is used between 5% and 70%, or between 10% and 60% by weight based on the total weight of the emulsion.

Oil-miscible co-solvent: According to the present invention, the oil phase in an emulsion of the invention may comprise a co-solvent such as, for example, undecane and tridecane (CETIOL ULTIMATE), C15-C19 alkanes, squalene, silicone oils, glycol ethers such as tripropylene glycol methyl ether (Dowanol™ TPM), dipropylene glycol n-propyl ether (Dowanol™ DPnP), DIPG monomethyl ether, dimethyl adipate/dimethyl glutarate (FlexiSolv® DBE®-LVP esters), benzyl benzoate and mixtures thereof. In an embodiment, the oil-miscible co-solvent is undecane and tridecane (CETIOL ULTIMATE), tripropylene glycol methyl ether (Dowanol™ TPM), dipropylene glycol n-propyl ether (Dowanol™ DPnP), benzyl benzoate and mixtures thereof. When present, the oil-miscible co-solvent is used between 1% and 50%, or between 5% and 40% by weight based on the total weight of the emulsion.

Oil-miscible weighting agent: According to the present invention, the oil phase in the emulsion of the invention may comprise a weighting agent such as, for example, gums (such as estergum, damargum), and/or sucrose acetate isobutyrate (SAIB). In some aspects, the weighting agent in the oil phase is estergum or damargum. When present, the oil-miscible weighting agent is used between 5% and 50%, or between 5% and 30% by weight based on the total weight of the emulsion.

Oil-miscible viscosifier: According to the present invention, the oil phase in the emulsion of the invention may comprise a viscosifying agent such as, for example, a cellulose derivative such as ethylcellulose, polyethylmethacrylate polymer, bis-isobutyl PEG-24/PPG-7/dimethicone copolymer and mixtures thereof. In some aspects, the oil viscosifier is ethylcellulose. When present, the oil-miscible viscosifier is preferably used between 1% and 30%, preferably between 2% and 10% by weight of the oil phase.

Water-miscible co-solvent: According to the present invention, an emulsion composition of the invention may comprise a water-miscible co-solvent, such as, for example, mono- and polyhydric solvents. Non limiting examples of such solvents include a C2-C4 alcohol, ethanol, propylene glycol, hexylene glycol, dipropylene glycol, glycerol, isopropylidene glycerol, butylene glycol (1,3-butanediol), 1,2-pentanediol, 1,2-hexanediol, 1,3-propanediol, and isopropanol, ethyl lactate and mixtures thereof. In some aspects, the water-miscible co-solvent is glycerol, 1,2-hexanediol and mixtures thereof. When present, the water-miscible co-solvent is used between 1% and 50%, or between 2% and 20% by weight of the water phase.

Water-miscible viscosifier: According to the present invention, a water phase of the emulsion in the invention may comprise a viscosifying agent such as xanthan gum, a natural gum (such as agar gum, alginic acid, gellan gum, tragacanth gum), guar gum, cellulose derivatives, polyacrylic acid, and/or an acrylate copolymer. In some aspects, the viscosifying agent in water is xanthan gum, gellan gum, polyacrylic acid, acrylate copolymer or a mixture thereof. When present, the viscosifier in water is used between 0.05% and 5%, between 0.05% and 1%, or between 0.05% and 0.3% by weight of the water phase.

Optional ingredients: According to the present invention, an emulsion composition may further comprise optional ingredients such as colorants, preservatives, opacifiers, emollients, humectants, antioxidants, free radical scavengers, POV remediation agents, cooling agents, vitamins, insect repellents, fixatives, cosmetic benefit agents, chelators, functional polymers, electrolytes, and pH adjusters.

Suitable cooling agents include, for example, menthol; menthone; isopulegol; N-ethyl-p-menthanecarboxamide; N,2,3-trimethyl-2-isopropylbutanamide; ethyl N-[[5-methyl-2-(isopropyl)cyclohexyl]carbonyl]glycinate; menthyl lactate; menthone glycerine acetal; mono-menthyl succinate; mono-menthyl glutarate; O-menthyl glycerine; 2-sec-butylcyclohexanone; menthane; camphor; pulegol; cineol; mint oil; peppermint oil; spearmint oil; eucalyptus oil; 3-1-menthoxypropane-1,2-diol; 3-1-menthoxy-2-methylpropane-1,2-diol; p-menthane-3,8-diol; 2-1-menthoxyethane-1-ol; 3-1-menthoxypropane-1-ol; 4-1-menthoxybutane-1-ol, N-ethyl-p-menthyl-3-carboxiamide, isopulegol, cubebol, 1-menthol, 3-(L-menthoxy) propane-1,2-diol, cis/trans-p-menthane-3,8-diol, 3-(L-menthoxy)-2-methylpropane-1,2-diol, 2-[2-(p-menthan-3-yloxy)ethoxy]ethanol, menthoxyethanol, (1R,2R,4R)-1-(2-hydroxy-4 methylcyclohexyl)ethenone, (1R,2R,5R)—N-ethyl-5-methyl-2-(prop-1-en-2-yl)cyclohexanecarboxamide, N-(3-hydroxy-4-methoxyphenyl)-2-isopropyl-5,5-dimethyl-cyclohexanecarboxamide, N-[4-(cyanomethyl)phenyl]-2-isopropyl-5,5-dimethyl-cyclohexanecarboxamide, N-(2-Hydroxy-2-phenylethyl)-2-isopropyl-5,5-dimethyl-cyclohexane-1-carboxamide, L-menthol lactate, (−)-menthyl ethylene glycol carbonate, (−)-menthone 1,2-glycerol ketal, D/L-menthone 1,2-glycerol ketal, (−)-menthyl 1-propylene glycol carbonate, (−)-menthyl 2-propylene glycol carbonate, D/L-menthyl 1-propylene glycol carbonate, D/L-menthyl 2-propylene glycol carbonate, (1R,2S,5R)—N-(4-methoxyphenyl)-5-methyl-2-(1-methylethyl)cyclohexanecarboxamide, menthyl ethylamido oxalate, (E)-3-benzo[1,3]dioxol-5-yl-N,N-diphenyl-2-propenamide, 3,4-methylenedioxycinnamic acid, N,N-diphenylamide, N,N-dimethyl (−)-menthyl succinimide, (1R,2S,5R)—N-(4-(carbamoylmethyl)phenyl)-menthylcarboxamide, (−)-menthyl pyrrolidone carboxylate, L-phenylephrine p-menthane carboxamide, (1R,2S,5R)—N-(4-(cyanomethyl)-phenyl)menthylcarboxamide, (1R,2S,5R)—N-(2-(pyridin-2-yl)ethyl)menthylcarboxamide, 6-isopropyl-3,9-dimethyl-1,4-dioxaspiro[4.5]decan-2-one, (1R,2R,4S)-dihydroumbellulol, N-ethyl-p-menthane-3-carboxamide, 2-isopropyl-N,2,3-trimethylbutyramide, ethyl 3-(p-menthane-3-carboxamido)acetate, N-ethyl-2,2-diisopropylbutanamide, N-(1,1-dimethyl-2-hydroxyethyl)-2,2-dimethylbutanamide, N-cyclopropyl-5-methyl-2-isopropyl-cyclohexanecarbonecarboxamide, (−)-menthyl acetoacetate, (−)-menthyl succinate, (−)-menthyl (S)-3-hydroxybutyrate, (−)-menthyl glutarate, 1-[2-hydroxyphenyl]-4-[2-nitrophenyl]-1,2,3,6-tetrahydropyrimidine-2-one, di-(−)-menthyl glutarate, and N-(2-hydroxyethyl)-2,3-dimethyl-2-isopropylbutanamide.

Suitable fixatives include, for example, caprylyl alcohol, octanol, butyloctanol, isotridecyl alcohol, hexyldecanol, isocetyl alcohol, isostearyl alcohol, octyldecanol, octyldodecanol, decyltetradecanol, tetradecyloctadecanol, neopentyl glycol diethylhexanoate, PPG-3 myristyl ether, and PPG-20 methyl glucose ether.

Non-limiting examples of suitable insect repellants include citronella, dimethyl phthalate and n, n-dimethyl-m-tolumide.

As used herein, the term “peroxide value” or “POV” refers to the amount of equivalents of oxidizing potential per 1 kilogram of material. Without intending to be limited to any particular theory, the POV of a material is determined analytically. The term “POV” does not refer to a chemical compound or group of compounds, but may be used interchangeably with the products of autoxidation within a sample that cause a response during a POV test. These autoxidation products differ depending upon the particular material being tested. Many classes of chemical compounds will produce a response during a POV test, including but not limited to organic and inorganic hydroperoxides, organic and inorganic peroxides, peroxyhemiacetals, peroxyhemiketals, and hydrogen peroxide itself.

By way of illustration, one POV test is an iodometric oxidation-reduction titration. All POV-responsive compounds share the property that they are capable of oxidizing the iodide ion to molecular iodine within the time period specified for the test; in fact, the iodide oxidation reaction is the basis for the test. Thus, “POV” is a numerical value that represents the molar sum total of the all the iodide-oxidizing species in a particular sample. See, U.S. Pat. No. 10,456,339; U.S. Patent Publication No. 20200289388; WO 2020089440.

A “POV remediant” according to the present invention is an agent that reduces the POV in a sample.

The POV remediant may be an α-oxocarboxylic acid such as, for example, alpha keto glutarate, pyruvic acid, 2-oxovaleric acid, phenylglyoxylic acid, 2-oxobutyric acid, 2-oxo-2-furanacetic acid, oxaloacetic acid, α-ketoglutaric acid, 2-oxopentandioate, indole-3-pyruvic acid, 2 thiopheneglyoxylic acid, trimethylpyruvic acid, 2 oxoadipic acid, 4 hydroxyphenylpyruvic acid, phenylpyruvic acid, 2 oxooctanoic acid, or a combination thereof. POV remediant may be a 2-hydroxyketone such as, for example, an alpha ketoglutarate salt, alpha keto glutarate dimethyldodecylamine, acetoin, erythrulose, dihydroxyacetone, 2-keto-D-gluconic acid hemicalcium salt hydrate, (1S,2S,5S)-(−)-2-hydroxy-2-pinanone, benzoin, p-anisoin, benzil, 2-hydroxy-3-oxo-hexadecane, or a combination thereof.

An emulsion according to the present invention may comprise a weighting agent. The weighting agent increases the density of the oil phase and reduces the creaming of oil droplets or the sedimentation of water droplets in solution. A weighting agent according to the present invention may be, for example, a gum (such as estergum, damargum), and/or sucrose acetate isobutyrate (SAIB).

Thus, for example, an emulsion of the present invention may include 1% to 50% by weight of the emulsion of a weighting agent (e.g., Damar gum), or 3% to 25% by weight of the emulsion of the weighting agent (e.g., Damar gum).

An emulsion according to the present invention may include a moisturizing agent such as, for example, glycerol. In another aspect, an emulsion according to the present invention may include a texturizer such as, for example, xanthan gum, a natural gum (such as agar, alginic acid, gellan gum, tragacanth gum), polyacrylic acid, and/or an acrylate copolymer. An emulsion according to the present invention may further comprise a modifying agent such as, for example, lysine.

A cyclodextrin stabilizer of the present invention may be, for example, an α cyclodextrin, a β cyclodextrin, a γ cyclodextrin, a hydroxy-propyl modified cyclodextrin, a methyl modified cyclodextrin, or combinations thereof.

Oil ingredients may be added to the oil phase prior to emulsification to improve the touch of the emulsion onto the skin (fine fragrance and body care applications).

A moisturizing agent such as glycerol may be added to the water prior to emulsification to increase the viscosity of the water phase (enhanced stability) and provide a moisturizing effect on the skin upon application (fine fragrance and body care applications). Thus, for example, glycerol may constitute from 3% to 40% by weight of the emulsion, or from 3% to 20% by weight of the emulsion.

A viscosifying agent such as xanthan gum may be added to the water after emulsification to reduce the creaming or sedimentation of oil droplets in emulsion. For example, a viscosifying agent may constitute from 0.05% to 5% by weight of the emulsion, or from 0.1% to 2% by weight of the emulsion.

A modifying agent such as lysine is optionally used to adsorb at the surface of particles and give the particles greater affinity for the oil/water interface. Unless otherwise indicated, the terms “modifying agent” and “surface modifying agent” are used interchangeably and refer to agents that modify the surface of the stabilizer particles. The surface of the particles may be modified to modulate the surface polarity and thus the adsorption properties at the oil/water interfaces.

In some aspects, the emulsion comprises equal to or less than 1% of surfactant, relative to the amount of stabilizer, typically cyclodextrin. In other aspects, the emulsion is free of surfactant.

According to the present invention, water is 10% to 90% w/w of the emulsion. In one aspect, water is 10% to 60% w/w of the emulsion.

According to the present invention, oil is 10% to 90% w/w of the emulsion. In one aspect, oil is 20% to 80% w/w of the emulsion.

Fragrance oil is 1% to 50% w/w of emulsion. In an aspect of the present invention fragrance oil is 5% to 20% w/w of the emulsion.

According to the present invention, cyclodextrins are 0.01% to 5% w/w of the emulsion. In an aspect, cyclodextrins are 0.01% to 2% w/w of the emulsion.

According to the present invention, the weighting agent is 1% to 70% w/w of the emulsion. In an aspect, the weighting agent is 1% to 55% w/w of the emulsion.

Optional oil ingredients may comprise 1% to 50% w/w of the emulsion. In an aspect of the present invention, optional oil ingredients comprise 3% to 25% w/w of the emulsion.

Process:

Particles are dispersed in a water phase using strong shear (UltraTurrax 24000 rpm or ultrasonic probe for 30 s). Oil phase ingredients are mixed together separately. The oil phase is then added to the water phase and an emulsion is obtained under strong shear (UltraTurrax 24000 rpm for 2 min or ultrasonic probe for 30 s) if droplets of small size are targeted. Low shear (manual shaking or vortex for 1 min) is used to get giants oil droplets (>0.5 mm) suspended in water phase. The emulsion thus obtained can be the final product according to the invention. Optionally, a concentrated aqueous gel of a viscosifier (Xanthan gum for example) is prepared separately, and the emulsion is diluted in this concentrated gel to produce a final product according to the invention.

The particles are dispersed in the water phase under strong shear prior to adding the oil and proceed to emulsification to favor the formation of a direct O/W emulsion. The oil phase is prepared separately before addition to the water phase for emulsification processing.

The average size of the oil droplets obtained in the final product may be in the range of 1 um to 8 mm. More specifically, the average size of the oil droplets in the final product is in the range of 0.2 mm to 5 mm. In an aspect of the present invention, the oil droplets in the final product are visible by the naked eye.

An emulsion of the present invention may be prepared under low shear (manual or mechanical shear) to quickly obtain a large size of droplets.

Optionally, a viscosifying agent is solubilized in water or a water phase before adding the emulsion to it under low shear to produce the final product.

### EXAMPLES

### 1. Example 1: Composition and Process According to the Invention

a) Composition

| Ingredients | Content (% wt) | Function |
|---|---|---|
| β-cyclodextrin ¹⁾ | 0.5 | Stabilizer |
| Fragrance A ²⁾ | 19 | Perfume |
| Tributyl-O-acetylcitrate ³⁾ | 57.2 | Weighting agent |
| Undecane (and) tridecane ⁴⁾ | 3.8 | oil-miscible co-solvent |
| DI water | 19.5 | Continuous phase |

¹⁾ Cavamax ® W7; origin: Wacker
²⁾ Fragrance A (DiPG free); origin: Firmenich
³⁾ Tributyl-O-acetylcitrate; origin: Aldrich
⁴⁾ Cetiol Ultimate; origin: BASF

b) Process

In a 20 ml glass vial, 0.05 g of β-cyclodextrin was weighed and completed to 2 g with water. 2 g of a mix Fragrance A/Cetiol Ultimate/Tributyl-O-acetyl citrate=10/2/30 was added to the water phase and the mixture was manually shaken for 30 s and left at rest at room temperature. Then again 2 g the mix of oil was added to the mixture before manual shaking. The same procedure was repeated twice to get 8 g of the mix Fragrance A/Cetiol Ultimate/Tributyl-O-acetyl citrate=10/2/30 in the final solution.

c) Result

An emulsion with oil droplets of about 3 mm in size was obtained. Droplets were present from top to bottom of the sample to get finally an homogeneous solution with an appealing visual effect. The emulsion can be sprayed as is or manually shaken before use, to recover its initial aspect after shaking. The sample was stable for a few days.

### Example 2: Composition and Process According to the Invention

a) Composition

| Ingredients | Content (% wt) | Function |
|---|---|---|
| β-cyclodextrin ¹⁾ | 0.25 | Stabilizer |
| Fragrance A ²⁾ | 9.5 | Perfume |
| Tributyl-O-acetylcitrate ³⁾ | 28.6 | Weighting agent |
| Undecane (and) tridecane ⁴⁾ | 1.9 | oil-miscible co-solvent |
| DI water | 59.5 | Continuous phase |
| Acrylate copolymer ⁵⁾ | 0.25 | Viscosifying agent |

¹⁾ Cavamax ® W7; origin: Wacker
²⁾ Fragrance A (DiPG free); origin: Firmenich
³⁾ Tributyl-O-acetylcitrate; origin: Aldrich
⁴⁾ Cetiol Ultimate; origin: BASF
⁵⁾ Carbopol ® Aqua SF-1; origin: Lubrizol

b) Process

The emulsion described in example 1 was prepared. Separately, a 0.5% gel of Carbopol® Aqua SF-1 was prepared in water and the pH was adjusted to 6 using NaOH (0.1%) and HCl (0.1%). The emulsion described in example 1 was diluted twice in the aqueous gel under gentle manual shaking to get a viscosified emulsion.

c) Result

An emulsion with oil droplets of about 3 mm in size was obtained. Droplets were present from top to bottom of the sample to get finally an homogeneous solution for several days at room temperature with an appealing visual effect. Emulsion can be sprayed as is or manually shaken before use, to recover slowly its initial aspect after shaking.

### Example 3: Composition and Process According to the Invention

a) Composition

| Ingredients | Content (% wt) | Function |
|---|---|---|
| β-cyclodextrin ¹⁾ | 0.125 | Stabilizer |
| Fragrance A ²⁾ | 4.75 | Perfume |
| Tributyl-O-acetylcitrate ³⁾ | 14.3 | Weighting agent |
| Undecane (and) tridecane ⁴⁾ | 0.95 | oil-miscible co-solvent |
| DI water | 79.625 | Continuous phase |
| Acrylate copolymer ⁵⁾ | 0.25 | Viscosifying agent |

¹⁾ Cavamax ® W7; origin: Wacker
²⁾ Fragrance A (DiPG free); origin: Firmenich
³⁾ Tributyl-O-acetylcitrate; origin: Aldrich
⁴⁾ Cetiol Ultimate; origin: BASF
⁵⁾ Carbopol ® Aqua SF-1; origin: Lubrizol

b) Process

The emulsion described in example 1 was prepared. Separately, a 1% gel of Carbopol® Aqua SF-1 was prepared in water and the pH was adjusted to 6 using NaOH (0.1%) and HCl (0.1%). The emulsion described in example 1 was 4× diluted in the aqueous gel under gentle manual shaking to get a viscosified emulsion.

c) Result

An emulsion with oil droplets of about 3 mm in size is obtained. Droplets are present from top to bottom of the sample to get finally an homogeneous solution for several days at room temperature with an appealing visual effect. Emulsion can be sprayed as is or manually shaken before use, to recover slowly its initial aspect after shaking.

### Example 4: Composition and Process According to the Invention

a) Composition

| Ingredients | Content (% wt) | Function |
|---|---|---|
| β-cyclodextrin ¹⁾ | 0.625 | Stabilizer |
| Fragrance A ²⁾ | 37.5 | Perfume |
| Tributyl-O-acetylcitrate ³⁾ | 37.5 | Weighting agent |
| DI water | 79.625 | Continuous phase |

¹⁾ Cavamax ® W7; origin: Wacker
²⁾ Fragrance A (DiPG free); origin: Firmenich
³⁾ Tributyl-O-acetylcitrate; origin: Aldrich

b) Process

In a 20 ml glass vial, 0.05 g of β-cyclodextrin was weighed and completed to 2 g with water. 2 g of a mix Fragrance A/Tributyl-O-acetyl citrate=50/50 was added to the water phase and the mixture was manually shaken for 30 s and left at rest at room temperature. Then again 2 g the mix of oil was added to the solution before manual shaking. The same procedure was repeated again to get 6 g of the mix Fragrance A/Cetiol Ultimate/Tributyl-O-acetyl citrate=50/50 in the final solution.

c) Result

An emulsion with oil droplets of about 3 mm in size was obtained. Droplets were present from top to bottom of the sample to get finally an homogeneous solution for several days at room temperature with an appealing visual effect. The emulsion can be sprayed as is or manually shaken before use, to recover slowly its initial aspect after shaking.

### Example 5: Composition and Process According to the Invention

a) Composition

| Ingredients | Content (% wt) | Function |
|---|---|---|
| β-cyclodextrin ¹⁾ | 0.5 | Stabilizer |
| Fragrance A ²⁾ | 25 | Perfume |
| Tributyl-O-acetylcitrate ³⁾ | 50 | Weighting agent |
| Undecane (and) tridecane ⁴⁾ | 5 | oil-miscible co-solvent |
| DI water | 19.5 | Continuous phase |

¹⁾ Cavamax ® W7; origin: Wacker
²⁾ Fragrance A (DiPG free); origin: Firmenich
³⁾ Tributyl-O-acetylcitrate; origin: Aldrich
⁴⁾ Cetiol Ultimate; origin: BASF

b) Process

In a 20 ml glass vial, 0.05 g of β-cyclodextrin was weighed and completed to 2 g with water. 2 g of a mix Fragrance A/Cetiol Ultimate/Tributyl-O-acetyl citrate=10/2/20 was added to the water phase and the mixture was manually shaken for 30 s and left at rest at room temperature. Then again 2 g the mix of oil was added to the solution before manual shaking. The same procedure was repeated twice to get 8 g of the mix Fragrance A/Cetiol Ultimate/Tributyl-O-acetyl citrate=10/2/20 in the final solution.

c) Result

An emulsion with oil droplets of about 3 mm in size was obtained. Droplets were present from top to bottom of the sample to get finally an homogeneous solution for several days at room temperature with an appealing visual effect. The emulsion can be sprayed as it or manually shaken before use, to recover slowly its initial aspect after shaking.

### Example 6: Composition and Process According to the Invention

a) Composition

| Ingredients | Content (% wt) | Function |
|---|---|---|
| β-cyclodextrin ¹⁾ | 0.25 | Stabilizer |
| Fragrance A ²⁾ | 12.5 | Perfume |
| Tributyl-O-acetylcitrate ³⁾ | 25 | Weighting agent |
| Undecane (and) tridecane ⁴⁾ | 2.5 | oil-miscible co-solvent |
| DI water | 59.5 | Continuous phase |
| Acrylate copolymer ⁵⁾ | 0.25 | Viscosifying agent |

¹⁾ Cavamax ® W7; origin: Wacker
²⁾ Fragrance A (DiPG free); origin: Firmenich
³⁾ Tributyl-O-acetylcitrate; origin: Aldrich
⁴⁾ Cetiol Ultimate; origin: BASF
⁵⁾ Carbopol ® Aqua SF-1; origin: Lubrizol

b) Process

The emulsion described in example 5 was prepared. Separately, a 0.5% gel of Carbopol® Aqua SF-1 was prepared in water and the pH was adjusted to 6 using NaOH (0.1%) and HCl (0.1%). The emulsion described in example 5 was diluted twice in the aqueous gel under gentle manual shaking to get a viscosified emulsion.

c) Result

An emulsion with oil droplets of about 3 mm in size was obtained. Droplets were present from top to bottom of the sample to get finally an homogeneous solution for several days at room temperature with an appealing visual effect. The emulsion can be sprayed as is or manually shaken before use, to recover slowly its initial aspect after shaking.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

1. An emulsion comprising:
(a) an aqueous phase comprising water,
(b) an oil phase comprising a perfume oil and an oil-miscible solvent, and
(c) a stabilizer selected from the group consisting of: an α cyclodextrin, a β cyclodextrin, a γ cyclodextrin, a hydroxy-propyl modified cyclodextrin, a methyl modified cyclodextrin, or combinations thereof.

2. The emulsion of claim 1, wherein the oil-miscible solvent in selected from the group consisting of: tributyl-O-acetylcitrate, triethylcitrate, caprylic triglyceride, triacetin, coconut alkanes (and) coco-caprylate/caprate, propanediol dicaprylate, octanoic acid 1,3-propanediyl ester, isopropyl palmitate, isopropyl myristate, ethyl oleate, triheptanoin, caprylic/capric glycerides, and mixtures thereof.

3. The emulsion of claim 1, wherein the oil-miscible solvent is between 1% and 70% by weight based on the total weight of the emulsion.

4. (canceled)

5. The emulsion of claim 1, further comprising an oil-miscible co-solvent.

6. The emulsion of claim 5, wherein the oil-miscible co-solvent is selected from the group consisting of: undecane and tridecane, C15-C19 alkanes, squalene, a silicone oil, a glycol ether such as tripropylene glycol methyl ether, dipropylene glycol n-propyl ether, DIPG monomethyl ether, a dimethyl adipate/dimethyl glutarate ester, benzyl benzoate and mixtures thereof.

7. The emulsion of claim 5, wherein the oil-miscible co-solvent is between 1% and 50% by weight based on the total weight of the emulsion.

8. (canceled)

9. The emulsion of claim 1, wherein the α cyclodextrin, β cyclodextrin, γ cyclodextrin, hydroxy-propyl modified cyclodextrin, methyl modified cyclodextrin, or combination thereof is between 0.001% and 10% by weight of the total emulsion.

10. (canceled)

11. (canceled)

12. The emulsion of claim 1, wherein the water phase does not include a C2-C4 alcohol.

13.-16. (canceled)

17. The emulsion of claim 1 wherein the water phase further comprises an oil-miscible viscosifier.

18. The emulsion of claim 17, wherein the oil-miscible viscosifier is selected from the group consisting of: a cellulose derivative such as ethylcellulose, polyethylmethacrylate polymer, bis-isobutyl PEG-24/PPG-7/dimethicone copolymer and mixtures thereof.

19. The emulsion of claim 17, wherein the oil-miscible viscosifier is between 1% and 30% by weight of the oil phase.

20. (canceled)

21. The emulsion of claim 1, wherein the water phase further comprises a water-miscible viscosifier.

22. The emulsion of claim 21, wherein the water-miscible viscosifier is selected from the group consisting of: xanthan gum, a natural gum, guar gum, a cellulose derivative, polyacrylic acid, an acrylate copolymer, and a combination thereof.

23. The emulsion of claim 21, wherein the water-miscible viscosifier is between 0.05% and 5%, by weight of the water phase.

24.-28. (canceled)

29. The emulsion of claim 1, wherein the emulsion is a water-in-oil or an oil-in-water emulsion.

30. The emulsion of claim 29 wherein the water phase is droplets dispersed in the oil phase and comprises from about 10% to about 80 wt % of the emulsion.

31. The emulsion of claim 29, wherein the oil phase is droplets dispersed in the water phase and comprises from about 10% to about 80 wt % of the emulsion.

32. The emulsion of claim 1, wherein the perfume oil in the oil phase is from 1% to 50 wt % of the emulsion.

33.-42. (canceled)

43. The emulsion of claim 1 wherein the emulsion comprises equal to or less than 1% of surfactant, relative to the amount of the stabilizer.

44. (canceled)

45. A consumer product comprising the emulsion of claim 1, wherein the consumer product is a fine fragrance product, a body care product, or an air care product.

46. (canceled)
